(19) United States
(12) Patent Application Publication
(10) Pub. No.: US 2005/0163845 A1
(43) Pub. Date: Jul. 28, 2005

(54) System for the controlled release of active ingredients

(76) Inventors: Ubaldo Conte, Busto Arsizio (IT); Lauretta Maggi, Pavia (IT)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/507,345

(22) PCT Filed: Mar. 12, 2003
(86) PCT No.: PCT/EP03/02537

(30) Foreign Application Priority Data
Mar. 12, 2002 (IT) MI2002A000515

(51) Int. Cl. A61K 9/24

(52) U.S. Cl. 424/472

(57) ABSTRACT

A new release system is disclosed for releasing one or more active substances vehicularised in it, at a predetermined rate and duration. The system is constituted by a pharmaceutical tablet, coated with a film of polymeric material impermeable and insoluble in aqueous fluids; on the coating film, one or more apertures of geometrically precise size and shape are made, through the use of a laser beam of appropriate power and intensity, in a way such that the active ingredient contained in the therapeutic system is released only from the non coated portion of the tablet surface at the desired rate and time. The technology for manufacturing such system is also described.

### STATE OF THE ART

The release of an active substance in a specific site of action and the possibility of releasing such active substances with a rate programmable “a prion” and in a time interval pre-determined by means of appropriate in vitro control tests, certainly constitutes one of the major sectors of development in pharmaceutical technology. Research of this type has in fact been carried out and continues to be carried out in many applicative sectors, including the controlled release of drugs, of dietary supplements, and of anti parasitic agents, for the prolonged release of fertilisers, herbicides, insecticides, snail/slug killers and/or specific protection agents for certain cultures in the agricultural and veterinary sector. Certainly however, the most innovative research sector in this field is that of drugs for human use.

The always difficult research for new active molecules (New Chemical Entities), characterised by very expensive investments at very high risk, has induced, above all in the last decade, the pharmaceutical industry to study and develop new systems and devices for releasing known active substances, at controlled and programmable rate. This approach allows to obtain sometimes a surprising improvement of the therapeutic effects.

In addition, these new drugs delivering systems and devices are particularly well accepted by patients, in that they allow targeted therapies simplification of the dosing scheme and notable benefits for the patients who can use, above all for the therapies of chronic diseases, drugs with dosing which can be reduced to a single administration per day, thus notably increasing the compliance of the subject, above all in the case of elderly people.

One of the fundamental innovative aspects of the pharmaceutical forms and/or of the controlled release systems is also the possibility of targeting the release of the drug (or of the active substance) in a specific site of action and to free such active substances with at a rate programmable through appropriate in vitro control test.

In fact, one of the most developed sectors has been that of the pharmaceutical forms and therapeutic systems able to release the active substance at a constant rate over a programmed time interval. It is evident that vehiculation of an active ingredient in a system capable of controlling the rate of the in vivo release brings about many therapeutic advantages such as maintaining a constant plasma concentration inside the therapeutic window for a prolonged period of time thus avoiding the fluctuations of the plasma levels associated with repeated administrations of conventional (rapid release) pharmaceutical forms and reducing the side effects and the undesired manifestations.

Amongst the pharmaceutical forms for oral administration, one of the most difficult problem to solve in the field of modified release of drugs (controlled and slowed), is represented by the control of the release of active ingredients or active substances having high solubility in aqueous environment.

In the field of pharmaceuticals, there have been many patents describing the preparation of pharmaceutical forms able to release the active ingredient with zero order kinetics, i.e. at a constant rate over time, and for a programmed period of time.

In particular, many of the embodiments known and used in therapy are constituted, in their simplest embodiment, by a hydrophilic matrix containing a drug and appropriate polymeric excipients able to release the active ingredient in modulated form (i.e. in a slowed form). In such matrix systems the active ingredient is dispersed in a polymer matrix; and the mechanism of release of the active ingredient relies on the physical-chemical characteristics of the matrix components.

Such matrix systems can be subdivided into:
inert matrices
hydrophilic gelable matrices
hydrophilic erodible matrices

From a general point of view the rate of drug's release from a polymer matrix can be described by the first law of Fick:

$$\frac{D M}{d t} = \frac{A D K Δ C}{h}$$

wherein
DM/dt=amount of drug released per unit of time
A=area available for release
D=diffusion coefficient
K=drug partitioning coefficient
ΔC=(C−Cs) concentration gradient between the two extremities of the layer to cross
h=thickness of the layer to cross

In the field of pharmaceuticals, the gelable and/or erodible hydrophilic matrices, manufactured by compression, are the most widely used modified release system for oral administration, thanks to the simplicity of production and the low costs. There are however some disadvantages and in particular the release of the vehicularised active ingredient does not take place at a constant rate but is variable over time: when the matrix comes into contact with aqueous fluids or biological liquids the rate of release is notably high and diminishes progressively until, when the system is running out, in the final stage, the release becomes excessively slow.

Following administration, from these systems plasma drug levels can be obtained at times even higher but not constant over time. This release mechanism can give rise to a “burst effect” in the initial stage, provoking a rapid absorption and the possible rise of the same undesired side effects, that these systems should prevent. This phenomenon is directly connected to the intrinsic characteristics of the pharmaceutical form and to the surface area of the tablet exposed to release which is very wide at the beginning of the process and reduces progressively over time.

There are many examples of such pharmaceutical forms which are fully described in the literature, such as in L. F. Prescott and W. S. Nimmo “Novel drug delivery and its therapeutic application” J. Wiley—New York 1989 and in S. Dumitriu “Polysaccharides in medicinal applications” M. Dekker—New York 1996.

The pharmaceutical applications refer to dosage forms suitable for different administration routes such as oral, transdermal, vaginal, ocular. For their importance and vast use of the oral administration of drugs, the most numerous and diversified embodiments are those destined for the gastrointestinal route.

Besides the matrix systems, which constitute the simplest and easiest approach to modified release of active substances, technological research has developed more complex and sophisticated systems; amongst these, worthy of citation is the OROS system described in the U.S. Pat. No. 4,160,020. It deals with a system constituted by a nucleus containing the drug dispersed in an osmotic agent, coated with a rigid semi-permeable film acting like an osmotic membrane which allows the water to pass through but not the active ingredient dissolved therein. A small calibrated hole is made on the coating. When the system is immersed in an aqueous liquid or in a biological fluid the water is drawn inside the system generating a pressure (osmotic) which pushes the active substance, or better a concentrated solution of the active substance, to come out through the calibrated hole. When the system has reached equilibrium the rate of release is constant.

The above described system is however not easily realised above all in the case of molecules having scarce solubility; for example, an osmotic system suitable for vehicularising 90 mg of nifedipine (a scarcely soluble drug) would require an osmotic system of around 1300 mg constituted by a tablet of over 13 mm in diameter which is certainly not easy to administer and does not meet the compliance of the patients.

### SUMMARY OF INVENTION

We have now unexpectedly found a new pharmaceutical form for oral administration, able to release one or more active ingredients or biologically active substances, at constant rate and for a certain period of time predeterminable through appropriate in vitro tests, which allows to overcome the drawbacks described above for the systems known in the art, and in particular the inconveniences of the OROS system.

The morphological and functional characteristics of the present therapeutic system will be illustrated in the following detailed description.

### BRIEF DESCRIPTION OF THE DRAWINGS

FIGS. 1 and 2: cross sectional views of the present tablet, showing the nucleus (1), the film coating (2) and the incision (3) delimiting an area of different dimensions.

### DETAILED DESCRIPTION OF THE INVENTION

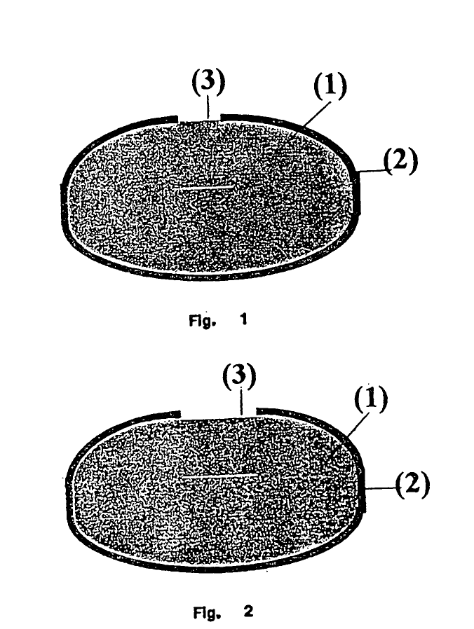

The release system according to the invention, as represented in FIGS. 1 and 2, is constituted by a pharmaceutical tablet comprising a nucleus coated with a film of polymeric material, insoluble and impermeable to water, characterised in that on said coating are made one or more incisions delimiting an area having exactly programmable and defined dimensions and geometric shape. Upon contact with water or with biological fluids, the portion of the coating film inside the incision(s) detaches and the active ingredient contained in the therapeutic system is released through that portion of the tablet surface that remained uncovered.

The incision(s) are preferably made by a laser beam.

The therapeutic system of the invention can be manufactured by the current manufacturing technologies, and therefore the system is immediately scalable to the industrial level, in particular as far as the system of the incision in the coating through the use of a laser beam is concerned.

The nucleus can be constituted of, in addition to the active substance, also other components, excipients and polymeric materials having different solubility, different hydrophilicity, different rates of hydration, erosion and/or gelation.

Usually such types of tablets are indicated as polymeric matrices with modified release and are preferably designed for the oral administration of biologically active substances.

The formulation of the tablet according to the present invention comprises hydrophilic and/or lipophilic polymeric materials in different percentages and is characterised in that the release of the active substance(s) takes place, as a function of the tablet structure, according to kinetics pre-programmable through appropriate in vitro tests.

One of the characteristics of the present tablet consists in the fact that in the preparation of the nucleus, in addition to the active ingredient(s), also polymeric substances capable of modulating (slowing and/or accelerating) the release of the active ingredient(s) are used.

As the active ingredients non steroid anti inflammatory drugs (NSAID) can be used, such as sodium dichlophenac, indomethacine, ibuprofen, ketoprofen, diflunisal, pyroxicam, naproxene, flurbiprofen, sodium tolmethin, or steroid anti inflammatory or substances inducing sleep and tranquillisers such as diazepam, nitrazepam, flurazepam, oxazepam, chlordiazepoxide, medazepam, lorazepam or active ingredients for the control of hypertension, for example Ace inhibitors or calcium antagonists such as enalapril, nifedipine, nitrendipine, nicardipine or diltiazem, propranolol, atenolol, pindolol, prazosin, ramipril, spirapril, spironolactone, methypranolol, molsidomine, moxonidine, nadolol, nadoxolol, levodopa, metoprolol, timolol or anti histaminic and/or antiasthmatic drugs such as ephedrine, terphenadine, teofilline, chlorpheniramine, or beta lactamic antibiotics, alone and in association, and derivatives thereof such as ampicillin, amoxicillin, cefradine, salts or derivatives thereof, and in particular in association with inhibitors of beta lactamase such as clavulanic acid.

Inside said polymeric matrix can be vehicularised: active substances for the treatment of chronic diseases such as drugs active on the cardiovascular system, anti arhythmics, cardiac stimulants, vasodilators, anti hypertensives, anti adrenergic substances with central or peripheral action or substances acting on the smooth arteriolar musculature, substances acting on the renin-angiotensin system, anti hypertensives and diuretics in association, diuretics anti Parkinson drugs for the treatment of Alzheimer's and Parkinson's diseases.

The preparation of the tablets (nucleus) can be carried out by the usual compression techniques of the mixture of powders or granulates, operating at pressures ranging between 1000 and 5000 Kg/cm².

Polymeric substances of possible use in the preparation of said matrix (or nucleus) are for example polyvinylpyrrolidone, hydroxypropylmethylcellulose with molecular weights from 2.000 to 4.000.000, sodium carboxymethylcellulose, carboxymethylamide, potassium methacrylate-divinylbenzene copolymer, polyvinylalcohols, hydroxypropylcellulose with molecular weights from 2000 to 4000000, polyoxyethylene (PEO) of molecular weight ranging between 100 and 10000000, carboxyvinylpolymers, polyvinylalcohols with molecular weight from 10000 to 1000000, glucans, scleroglucans, mannans, carragenans, galactomannans, gellans, xanthans, alginic acid and derivatives, polyanhydrides, polyaminoacids, poly-(methyl vinyl ethers/maleic anhydride) carboxymethylcellulose and derivatives, ethylcellulose, methylcellulose and cellulose derivatives in general, amides, starch derivatives, alpha, beta, gamma cyclodextrins and dextrine derivatives and copolymers of the above cited polymers.

Said polymeric substances constitute from 1% to 90% of the weight of the matrix (or nucleus), and preferably between 5% and 50%.

All cited polymers are commercially available in different pharmaceutically acceptable forms, characterised by different physical-chemical properties, and by different solubility and gelation properties.

In particular, as far as hydroxypropylmethylcellulose is concerned, various types of this polymer can be used, having different molecular weights (from 1000 to 4000000) and different degrees of substitution. Said types of hydroxypropylmethylcellulose have differentiated characteristics, and are prevalently erodible or prevalently gelable, according to the viscosity or to the degree of substitution (D.S.) in the polymeric chain.

In the nucleus substances which facilitate the disintegration of agglomerates can be also find use, such as the disintegrants commonly used in the pharmaceutical field and well known to any person skilled in the art and/or the super disintegrants, such as crosslinked polyvinylpyrrolidone, crosslinked sodium carboxymethylcellulose, sodium amidoglycolate, and microcrystalline cellulose.

In order to favour the release of the active substance, pharmaceutically acceptable effervescent mixtures, known to any person skilled in the art, can also be used.

A fundamental characteristic of the new embodiment is constituted by the fact that in addition to the previously cited hydrophilic polymers, in the formulation can find use lipophilic and/or amphiphilic substances, in which the hydrophilic portion can be represented by polyalcohols whilst the lipophilic part is represented by unsaturated or saturated fatty acids, in the form of hydrogenated vegetable oils.

The association of the hydrophilic portion with the lipid chain is obtained by esterification or partial alcoholysis of hydrogenated vegetable oils by PEG molecules or glycerol or other polyol. This way one obtains compounds having differing degrees of hydrophilicity which can be evaluated by the determination of the Hydrophylic-Lipophylic Balance (HLB) value. Triglycerides with HLB values between 1 and 2, diglycerides with HLB values between 2 and 3, monoglycerides with HLB values between 3 and 4, PEG diesters with HLB values between 6 and 15, PEG monoesters with HLB values between 10 and 17, triglycerides with HLB values between 1 and 2 are available. In practice, the higher is the HLB value, the greater is the hydrophilic character and, obviously, the lower is the lipophilic character.

Finally, excipients commonly used in pharmaceuticals can be used in the present nucleus, such as mannitol, lactose, sorbitol, xylitol, talc, stearic acid, sodium benzoate, magnesium stearate, colloidal silica and others such as gliceryl monostearate, hydrogenated castor oil, waxes, mono-, di- and tri-substituted glycerides, glicerylpalmitoylstearate, gliceryl behenate, cetyl alcohol.

When it is desired to favour the penetration of water and/or aqueous fluids into the nucleus, hydrophilic diluents are added, such as mannitol, lactose, starches of various origins, sorbitol, xylitol, wetting agents and/or agents favouring the penetration of water into the agglomerate are added to the formulation.

Instead, when it is desired to slow the penetration of water and/or aqueous fluids into the nucleus, hydrophobic diluents are added, such as glyceryl monostearate, hydrogenated castor oil, waxes and mono-, di- and tri-substituted glycerides.

In addition, diluents, binding agents, lubricants, buffers, anti adherents, glidants, plasticisers, and other substances able to confer to said layer the desired characteristics can be used in the nucleus as will be better explained in the examples reported below.

The tablet which constitutes the nucleus of the new release system according to the present invention is completely coated by film coating in a coating pan or by another industrially applicable procedure, with appropriate impermeable and insoluble coatings, which impede the release of the active ingredient from the coated surface.

For the coating of said tablets polymeric materials insoluble in water such as acrylates, methacrylates and ethylcellulose can find use.

The filming procedure can be carried out by the traditional method in a coating pan, or in a perforated basin or in a fluidised bed according to processes known to any person skilled in the art.

To obtain a homogeneous coating applied uniformly on the whole surface of the tablet, in the composition of the coating solution or suspension, are used plasticising substances such as triethylcitrate, ethyl phthalate, butyl phthalate, diethylsebacate, propylene glycol, polyoxyethyleneglycols with different molecular weights, castor oil.

In addition are also used colouring agents and/or substances altering opacity according to the characteristics of the coating.

Said coating, prior to the operation of the incision by laser beam, constitutes from 0.2 to 30% of the tablet weight, and preferably from 2 to 25%.

Only onto the coating are made one or more incisions delimiting an area having predetermined geometric shape and exactly controlled dimensions; the incision(s) can be made on any part of the coating of the tablet, preferably on one face, and have dimensions ranging from 2% to 80%, and preferably from 5% to 70% of the total surface of the coating.

The coating is removed by the use of a laser beam which creates a precise incision of predetermined geometric shape (in the most simple case a circle) and of an area defined with extreme precision so that only the coating is cut without touching the underlying section of the tablet. The incision in the coating by laser beam can be carried out on one or both faces of the tablet, making a single incision or more incisions so as to obtain a free surface area, able to release the active substance, at the desired rate and in a pre-determined time interval, by interaction with the means of dissolution.

The incision in the coating allows the penetration of dissolution fluid. The contact with water or with the biological fluid determines the beginning of the erosion and/or of the slow gelation of the constituents of the matrix system with the consequent lifting of the film portion inside the incision, the coating detaches and provokes the exposure of the surfaces of the nucleus and therefore the full interaction between the means of dissolution and the nucleus containing the active substance.

The new controlled release system of the invention is therefore characterised in that the release of the active ingredient contained in the tablet by interaction with the means of dissolution can take place only through the hole(s) made in the coating and therefore at a rate that depends on the area of the exposed surface and not on the osmotic pressure.

More precisely, the dose of drug begins to be released only through the hole(s) made in the coating, at a rate that depends on the area of the exposed surface which is in contact with water or with biological fluids and therefore, in case the hole(s) are circular, it depends on the diameter of the hole(s).

According to a further embodiment of the present invention, on the filmed tablet having one or more incisions on the insoluble coating, as previously described, a second gastro-resistant and enterosoluble polymeric coating is applied, based for example on acrylic and methacrylic copolymers, cellulose aceto-phthalate, cellulose aceto-propionate, cellulose trimellitate and other natural, synthetic or semi-synthetic derivatives of cellulose, of hydroxypropylcellulose, of hydroxypropylmethylcellulose, for example hydroxypropylmethylcellulose acetate succinate.

This allows an additional control over release, because in the gastric environment the active substance vehicularised in the system is not released and the system is activated only at the enteric level, when the enterosoluble coating dissolves. The release of the vehicularised active ingredient begins only following the coating solubilisation and takes place only through the hole made in the coating of is impermeable polymeric material.

This configuration of the tablet allows the active substance to be released only at the enteric level, and can be used to obtain the release of drugs up to the distal portion of the enteric tract, for a release at the level of colon or the rectum.

Further object of the present invention is a procedure for the preparation of the present therapeutic system. Such a procedure includes the preparation of a coated tablet according to traditional techniques, known to any skilled person, followed by the incision of the coating of the tablet by the use of a laser beam.

In particular, the filmed tablets are positioned onto a horizontal plane and one face of the tablet is incised by exposure to a laser beam. The duration of exposure to the laser beam depends on the thickness of the coating and on the power of the laser apparatus. For example, if a laser apparatus having a power of 20 W is used, an exposure of 100 milliseconds is necessary to cut a coating having a thickness of 100 □m.

The release system of the invention has the advantage of releasing the active ingredient vehicularised in a programmed manner and it is therefore possible to vehicularise a reduced quantity of drug, with respect to the traditional delayed release forms, avoiding the phenomenon of dose dumping; therefore, the present release system having a controlled release of the active ingredient satisfies specific therapeutic requirements.

In the following examples, the morphological characteristics and functions of the present innovative therapeutic systems will be better illustrated. In particular, matrix systems containing a soluble active ingredient are prepared. Said matrix systems are then completely coated with an insoluble coating (for example based on ethylcellulose), onto which a second gastroresistant and enterosoluble coating (for example based on acrylic and methacrylic copolymer) can be applied, as it is known to any person skilled in the field.

The examples and the results obtained in the pharmaceutical forms described above, highlight better the conceptual and functional characteristics of the new system.

### EXAMPLE 1

### Preparation of a Series of (5000) Tablets Containing as the Active Ingredient 120 mg of Diltiazem

1.a—Composition of the Matrix

| | |
|---|---|
| Diltiazem HCl | 120.0 mg |
| Hydroxypropylmethylcellulose (Methocel ® E4M) | 60.0 mg |
| Lactose (FU) | 60.0 mg |
| Methylcellulose (Methocel ® A4) | 0.7 mg |
| Magnesium stearate (FU) | 2.0 mg |
| Colloidal silica (Siloyd ® 244) | 1.0 mg |
| Total | 243.7 mg |

The appropriate quantity of Diltiazem, lactose and hydroxypropylmethylcellulose (Methocel® E4M) are mixed in a V shaped mixer. The homogeneous mixture is wetted with an aqueous solution of methylcellulose. The humid mass is forced through a 25 mesh grid obtaining a granulate which is dried in an oven to constant weight, added with magnesium stearate and colloidal silica and then mixed in a V shaped mixer for 15 min.

The granulate thus obtained is used for the preparation of the tablets as reported in the following point 1.b.

1.b—Preparation of the Tablets

The granulate obtained according to above reported procedures and according to schemes well known to any skilled person, is loaded into the loading hopper of a rotary press (Piccola—Ronchi-Milan).

The machine press, fitted with rounded dies of 10.0 mm in diameter, is set so as to produce tablets of 243.7 mg containing the active ingredient (equal to 120 mg of diltiazem).

1.c—Filming Process

Percentage Composition of the Coating:

| | |
|---|---|
| Copolymer of acrylic and methacrylic acid | 18.50% |
| (Eudragit ® L 30 D Rohm Pharma, D) | |
| Talc (C. Erba, Milan, I)) | 5.60% |
| Triethylcitrate (C. Erba, Milan, I)) | 1.80% |
| Water | 74.10% |
| Total | 100.00% |

The filming process is carried out using a coating pan for rapid coating (Manesty Accela-Cota) spraying, through an “air-less” system, a 30% aqueous dispersion of the acrylic and methacrylic acid copolymer (Eudragit® L 30 D) in which triethylcitrate is dissolved.

Operating with an air entry temperature of around 40-50°, according to the technique known to every expert in the field, tablets are obtained, which are coated with a uniform film of the above reported polymeric material.

1.d—Incision of the Coating

The filmed tablets, obtained as described in the preceding point 1.c, are positioned on a horizontal plane so as to present one face exactly below the beam of laser light produced by a CO₂ laser apparatus having a power of 20 W.

Centrally on the surface (coating) of one face of the tablet, circular incision(s) are made, having a diameter of 5.0 mm or of 7.0 mm only on the coating.

The incision(s) are made in a time of approx. 100 thousandths of a second and effect a thickness of approx. 100 □m, equal to the thickness of the coating.

1.e—Dissolution Test

The release systems prepared and described above in the examples 1.c and 1.d, i.e. respectively non coated tablets and tablets with the coating cut circularly (with diameters respectively of 5.0 mm or 7.0 mm) have been studied to evaluate the characteristics of the release of the active ingredient.

The apparatus 2, paddle (USP XXII) operating at 100 r.p.m. and 1 l of hydrochloric acid at pH 1.0 as such dissolution fluid, are used. The release of the active ingredient is followed by UV spectrophotometric determination at 236 nm using an automatic sampling and reading system (Beckman). Upon contact with the dissolution fluid, the part of the coating which has been effected by the laser incision, is raised by the light swelling of the tablet, thus freeing a circular surface of the nucleus of diameter 5.0 mm (equal to an area of approx. 19.5 mm²) or 7.0 mm (equal to an area of approx. 38.5 mm²).

The results of the tests carried out are reported in Table I.

TABLE I

| | % release | % release | % release |
|---|---|---|---|
| Time (min) | non coated tablet | hole 19.5 mm² | hole 38.5 mm² |
| 30 | 19.6 | 0.7 | 1.9 |
| 60 | 32.8 | 2.4 | 4.0 |
| 120 | 48.7 | 4.7 | 8.7 |
| 240 | 74.2 | 9.1 | 19.8 |
| 360 | 91.0 | 12.6 | 27.2 |
| 480 | 100.7 | 16.2 | 34.5 |
| 720 | | 20.1 | 50.9 |
| 960 | | 28.7 | 64.3 |
| 1200 | | 42.0 | 78.9 |

As can be easily seen from the Table I it is evident that from the non filmed tablet the active ingredient is released in approx. 7-8 hours. With respect to the non filmed tablet, the tablets with the cut coating show a release of the active ingredient at a controlled rate. In particular, the active ingredient can be released at different rates as a function of the area of the hole made in the coating. With equal compositions, from the filmed tablet with the circular hole of 7.0 mm in the coating (equal to 38.5 mm²) the active ingredient is released at a greater rate with respect to the system with the hole of 5.0 mm. (equal to 19.5 mm²).

This result fully answers to the objectives of the invention. It should be underlined that from the non filmed tablet the active ingredient is released more quickly.

### EXAMPLE 2

### Preparation of a Series of 5,000 Tablets Containing as the Active Ingredient 120 mg of Diltiazem

In this formulation hydroxypropylmethylcellulose with lower molecular weight is used, which allows to obtain a different rate of release of the active ingredient with respect to that observed in Example 1.

2.a—Composition of the Matrix:

| | |
|---|---|
| Diltiazem HCl | 120.0 mg |
| Hydroxypropylmethylcellulose (Methocel ® E50) | 60.0 mg |
| Lactose (FU) | 60.0 mg |
| Polyvinylpyrrolidone (Plasdone ® K30 - I.S.P.) | 8.0 mg |
| Magnesium stearate (FU) | 1.0 mg |
| Colloidal silica (Siloyd ® 244) | 0.5 mg |
| Total | 249.5 mg |

The appropriate quantity of Diltiazem with lactose and hydroxypropylmethylcellulose (Methocel® E50) are mixed in a V shaped mixer for 15 min., obtaining a homogeneous mixture which is then humidified with a hydroalcoholic solution of polyvinylpyrrolidone. The humid mass is forced through a 35 mesh grid, obtaining a granulate which is dried in an oven to constant weight, then added with magnesium stearate and colloidal silica, and mixed in a V shaped mixer for 15 min.

The mixture thus obtained is used for the preparation of the tablets as reported in the following point 2.b.

2.b—Preparation of the Tablets

The granulate obtained as described above according to well known procedures, is loaded into the loading hopper of a rotary press (Piccola-Ronchi-Milan).

The machine press, fitted with rounded dies of 10.0 mm of diameter, is set so as to produce tablets of 249.5 mg containing the active ingredient (equal to 120 mg of diltiazem).

2.c—Filming Process

Percentage Composition of the Coating:

| | |
|---|---|
| Surelease ® (Colorcon ® U.K) | 70.00% |
| Water | 30.00% |
| Total | 100.00% |

The filming process is carried out using a coating pan for rapid coating (Manesty Accela-Cota) spaying, through an “air-less” system a 70% aqueous dispersion of Surelease®. This filmogenic dispersion is commercially available; it is an aqueous dispersion of ethylcellulose and contains diethylsebacate as a plasticiser and oleic acid as a stabiliser. The aqueous dispersion is diluted with water prior to use.

2.d—Removal of a Known Portion of the Coating

The coated tablets obtained as described above in example 2.c, are positioned on an horizontal plane so as to present one face exactly below the beam of the laser light produced by a CO₂ laser apparatus having a power of 20 W.

Centrally on the surface of one face of the tablet, circular incisions are made, having a diameter of 5.0 mm or of 7.0 mm only on the coating.

The incision is carried out in a time of approx. 100 thousandths of a second and effects a thickness of approx. 100 □m equal to the thickness of the coating.

2.e—Dissolution Test.

The release systems prepared and described in the example 2.c and 2.d, respectively non filmed tablets and tablets with the coating which have circular incisions of diameter of 5.0 mm or 7.0 mm on just the coating, are studied to evaluate the release characteristics of the active ingredient.

At this aim, the apparatus 2, paddle (USP XXII) operating at 100 r.p.m. and 1 l of hydrochloric acid at pH 1.0 as such dissolution fluid, are used. The release of the active ingredient is followed by UV spectrophotometric determination at 236 nm using an automatic sampling and reading system (Beckman). Upon contact with the dissolution fluid, the part of the coating which has been effected by the laser incision, is raised by the slight swelling of the tablet, thus freeing a surface of the nucleus respectively of 19.5 mm² or 38.5 mm². The results of the tests carried out are reported in Table II.

TABLE II

| | % release | % release | % release |
|---|---|---|---|
| Time (mm) | non coated tablet | hole 19.5 mm² | hole 38.5 mm² |
| 15 | 19.5 | 0 | 0 |
| 30 | 39.4 | 1.2 | 3.5 |
| 60 | 72.5 | 3.8 | 12.7 |
| 90 | 93.1 | 8.1 | 22.8 |
| 120 | 100.8 | 12.6 | 32.5 |
| 180 | | 23.3 | 50.7 |
| 240 | | 38.7 | 68.4 |
| 300 | | 59.5 | 88.2 |
| 360 | | 80.5 | 95.6 |
| 420 | | 93.8 | 96.0 |
| 480 | | 97.8 | 98.8 |
| 540 | | 98.3 | |
| 600 | | 99.4 | |

As one can easily see from the Table II, it is evident that from the non filmed tablets the active ingredient is released in approx. 2 hours. With respect to the non coated tablets, the tablets with the cut coating show a release of the active ingredient at a controlled rate. The active ingredient can be released at different rates as a function of the area of the hole made in the coating. At equal compositions, from the filmed tablet with the circular hole of 7.0 mm in the coating (equal to 38.5 mm²) the active ingredient is released at a greater rate with respect to the system with the hole of 5.0 mm. (equal to 19.5 mm²). This result fully answers to the objectives of the invention.

### EXAMPLE 3

### Preparation of a Series of (5.000) Tablets Containing as the Active Ingredient 180 mg of Diltiazem

3.a—Composition of the Matrix:

| | |
|---|---|
| Diltiazem HCl | 180.0 mg |
| Hydroxypropylmethylcellulose (Methocel ® E50) | 90.0 mg |
| Lactose (FU) | 90.0 mg |
| Polyvinylpyrrolidone (Plasdone ® K30 - I.S.P.) | 12.0 mg |
| Talc (FU) | 4.0 mg |
| Magnesium stearate (FU) | 2.0 mg |
| Colloidal silica (Siloyd ® 244) | 0.5 mg |
| Total | 378.5 mg |

The appropriate quantity of Diltiazem with lactose and hydroxypropylmethylcellulose (Methocel® E50) are mixed in a V shaped mixer for 15 min. A homogeneous mixture is obtained, which is humidified with a hydroalcoholic solution of polyvinylpyrrolidone. The humid mass is forced through a 25 mesh grid, obtaining a granulate which is dried in an oven to constant weight, added with talc, magnesium stearate and colloidal silica, and then mixed in a V shaped mixer for 15 min.

The mixture thus obtained is used for the preparation of the tablets as reported in the following example 3.b.

3.b—Preparation of the Tablets

The granulate obtained according to the above reported procedure and according to schemes well known to all the experts in the field, is loaded into the loading hopper of a rotary press (Piccola-Ronchi-Milano).

The machine press, equipped with rounded dies of 10.0 mm in diameter, is set up so as to produce tablets of 378.5 mg containing the active ingredient (equal to 180 mg of diltiazem).

3.c—Filming Process

Percentage Composition of the Coating:

| | |
|---|---|
| Copolymer of acrylic and methacrylic acid | 18.50% |
| (Eudragit ® L 30 D Rohm Pharma, D) | |
| Talc (C. Erba, Milan, I)) | 5.60% |
| Triethylcitrate (C. Erba, Milan, I)) | 1.80% |
| Water | 74.10% |
| Total | 100.00% |

The filming process is carried out using a coating pan for rapid coating (Manesty Accela-Cota) spraying, through an “air-less” system a 30% aqueous dispersion of the acrylic and methacrylic acid copolymer (Eudragit® L 30 D) in which triethylcitrate is dissolved.

The procedure is carried out with the air in entry at a temperature of approx. 40-50° C., according to a known technique, thus obtaining tablets which are completely coated by an uniform film of the above said polymer material.

3.d—Incision of the Coating

The coated tablets obtained as described above in example 3.c, are positioned on a horizontal plane so as to present one face exactly below the beam of the laser light produced by a CO₂ laser apparatus having a power of 20 W.

Centrally on the surface (coating) of one face of the tablet, circular incisions are made, having a diameter of 5.0 mm or of 7.0 mm only on the coating.

The incision is carried out in a time of around 100 thousandths of a second and effects a thickness of approx. 100 □m, equal to the thickness of the coating.

3.e—Dissolution Test.

The release systems prepared and described in examples 3.c and 3.d, respectively non filmed tablets and tablets with the coating having circular incisions of 5.0 mm or of 7.0 mm in diameter only on the coating, have been studied to evaluate the system characteristics in releasing the active ingredient. At this aim, the apparatus 2, paddle (USP XXII) operating at 100 r.p.m. and 1 l of hydrochloric acid at pH 1.0 as dissolution fluid, are used. The release of the active ingredient is followed by UV spectrophotometric determination at 236 nm using an automatic sampling and reading system (Beckman). Upon contact with the dissolution fluid, the part of the coating which had been effected by the laser abrasion, raises due to the slight swelling of the tablet, thus freeing a surface of the nucleus respectively of 19.5 mm² or 38.5 mm². The results of the tests carried out are reported in Table III.

TABLE III

| | % release | % release | % release |
|---|---|---|---|
| Time (mm) | non coated tablet | hole 19.5 mm² | hole 38.5 mm² |
| 60 | 44.3 | 5.5 | 10.2 |
| 120 | 77.9 | 15.1 | 23.8 |
| 180 | 98.8 | 24.6 | 40.1 |
| 240 | 99.6 | 38.0 | 57.9 |
| 300 | 99.8 | 50.8 | 72.0 |
| 360 | 100.2 | 66.0 | 85.3 |
| 420 | 101.0 | 77.2 | 93.0 |
| 480 | 101.0 | 87.0 | 98.8 |
| 600 | 101.0 | 100.1 | 100.3 |

As can be easily seen from the Table III, it is clear that from the non filmed tablet the active ingredient is released in approx. 3-4 hours. With respect to the non coated tablets, the tablets with the cut coating show a release of the active ingredient at a controlled rate. The active ingredient can be released at different rates as a function of the area of the hole made in the coating. At equal compositions, from the filmed tablet with a hole of 38.5 mm² in the coating the active ingredient is released at a greater rate with respect to the system with a hole of 19.5 mm².

This result fully answers to the objectives of the invention.

1. A therapeutic system for the oral administration of one or more active ingredients in form of a tablet, characterised by the fact of comprising a nucleus containing the active ingredient(s) completely coated by a film coating of polymeric material, insoluble and impermeable to aqueous fluids, on which have been made one or more incisions which define an area of predetermined dimensions and shape as a function of the rate and the progress of release of the active ingredient(s) desired, said release taking place from the area of the nucleus underlying the surfaces of the coating film delimited by the incision(s) which is removed when the therapeutic system comes into contact with aqueous fluids.

2. The therapeutic system according to claim 1 in which the nucleus comprises one or more polymeric substances able to modulate the release of the active ingredient.

3. The therapeutic system according to claim 2, in which said polymeric substances constitute between 1% and 90% in weight of said nucleus.

4. A therapeutic system according to claim 3, in which said polymeric substances constitute between 5% and 50% in weight of the said nucleus.

5. The therapeutic system according to claim 1, in which the nucleus further comprises one or more disaggregating and/or super disaggregating agents.

6. The therapeutic system according to claim 1, in which the nucleus further comprises one or more effervescent mixtures.

7. The therapeutic system according to claim 1, in which the nucleus further comprises hydrophilic diluents and/or wetting agents.

8. The therapeutic system according to claim 1, in which the nucleus further comprises hydrophobic diluents.

9. The therapeutic system according to claim 1 in which the nucleus further comprises one or more substances selected from binding agents, lubricants, buffers, antiadherents, glidants and plasticisers.

10. The therapeutic system according to claim 1 in which the insoluble coating film comprises one or more plasticising substances.

11. The therapeutic system according to claim 1 in which the insoluble coating film constitutes from 0.2% to 30% in weight of the weight of the tablet.

12. The therapeutic system according to claim 11 in which the insoluble coating film constitutes from 2% to 25% in weight of the weight of the tablet.

13. The therapeutic system according to 1 in which the incision(s) define an area of dimensions comprised of between 2% and 80% of the total surface of the coating film.

14. The therapeutic system according to claim 13 in which the incision(s) define an area of dimensions comprised of between 5% and 70% of the total surface of the coating film.

15. The therapeutic system according to claim 1, in which onto the insoluble coating film a second film of gastroresistant and enterosoluble polymeric coating is applied.

16. A process for the manufacture of a therapeutic system according to claim 1, characterised by the fact that the incision(s) in the coating film are made by laser.

17. The process according to claim 16, in which the incision(s) in the coating film are made with a CO₂ laser device having a power of 20 W.
